(19) United States
(12) Patent Application Publication
(10) Pub. No.: US 2012/0265265 A1
(43) Pub. Date: Oct. 18, 2012

(54) Automated External Defibrillator Pad System

(76) Inventors: Mehdi Razavi, (US); Renata F. Ramos, (US); Joanna Christabel Nathan, (US); Bradley John Otto, (US); Lingxiao Jiang, (US); Carl J. Nelson, (US); Justin Lin, (US)

(21) Appl. No.: 13/445,429

(22) Filed: Apr. 12, 2012

Related U.S. Application Data
(60) Provisional application No. 61/474,929, filed on Apr. 13, 2011.

(51) Int. Cl. A61N 1/39

(52) U.S. Cl. 607/7; 607/5

(57) ABSTRACT

An embodiment includes a cardiac resuscitation system with first and second electrodes in a source electrode pad and a return electrode in a return electrode pad. After the source and return electrode pads are applied to a patient the layperson may put a switch in a first position to create a first electrical path to communicate a first shock between the first and return electrodes via a first vector. If the first shock fails the user may move the switch to a second position to create a second electrical path to communicate a second shock between the second and return electrodes via a second vector. As a result, the system allows a layperson to easily flip a switch to produce a first shock via a first vector and a second shock via a second vector thereby improving therapy outcomes. Other embodiments are described herein.

This application claims priority to U.S. Provisional Patent Application No. 61/474,929 filed on Apr. 13, 2011 and entitled “Automated External Defibrillator Pad System to Redirect Electric Current During Cardiac Fibrillation, and a Method for Enabling Simple and Rapid Discharge Vector Changes for Automatic External Defibrillators,” the contents of which are hereby incorporated by reference.

### BACKGROUND

A heart normally functions via synchronized propagation of electrical activity through heart tissue. An arrhythmia occurs when this activity becomes irregular and/or desynchronized. Certain arrhythmias, such as ventricular fibrillation, can be life-threatening and must be treated quickly.

Automated External Defibrillators (AEDs) treat arrhythmias by transmitting a large current, via electrode pads placed on the patient's chest, through the thoracic cavity and across the heart. The current proceeds via a “vector” having a magnitude and a direction. If the current proceeds along the wrong vector it will not target the heart effectively and will not stop the arrhythmia. However, an effective current application (“shock”) proceeding along the proper vector proceeds across the heart and stops the arrhythmia. If the shock fails additional shocks may be needed. Trying to limit the number of shocks administered to the patient results in higher survival rates, decreased brain damage, fewer skins burns, and lower myocardial damage.

A shock may fail to terminate an arrhythmia due to the patient's condition. However, the shock may also fail because a user misapplied the electrode pads in such a way that the shock current proceeds at an improper vector that does not properly target the heart. Regardless of why a shock does not restore normal rhythm, a first shock may be unsuccessful in correcting the arrhythmia and a second shock, applied via another vector, may be needed. Placement of a second set of pads to apply a second shock via an alternate vector (in hopes the different vector will remedy the arrhythmia) may take 60 seconds or longer. Since an arrhythmia can lead to cardiac arrest within minutes, this delay can have dire results. Furthermore, the person operating the AED may be an untrained layman who is understandably stressed by the situation. As such, the operator may panic and be ineffective in applying a second shock, repositioning pads for a second shock via another vector, applying new pads for a second shock via another vector, and the like.

### BRIEF DESCRIPTION OF THE DRAWINGS

FIG. 1 includes a cardiac resuscitation system in an embodiment of the invention.

FIG. 2 includes a method for using a cardiac resuscitation system in an embodiment of the invention.

FIG. 3 includes an electrode pad in an embodiment of the invention.

FIG. 4 includes an electrode pad in an embodiment of the invention.

FIG. 5 includes a computer system for use with various embodiments of the invention.

FIG. 6 includes a source electrode pad in an embodiment of the invention.

FIGS. 7(a)(b)(c) illustrate testing conditions for an embodiment of the invention.

### DETAILED DESCRIPTION

In the following description, numerous specific details are set forth but embodiments of the invention may be practiced without these specific details. Well-known circuits, structures and techniques have not been shown in detail to avoid obscuring an understanding of this description. “An embodiment”, “example embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Also, while similar or same numbers may be used to designate same or similar parts in different figures, doing so does not mean all figures including similar or same numbers constitute a single or same embodiment.

An embodiment includes an AED or other cardiac resuscitation system with first and second surface electrodes included in a single source electrode pad and a return electrode included in a return electrode pad. The three electrodes may couple to the AED via a single connector so a layperson can quickly connect the electrodes to the AED using only a single connection. The system may include a switch. After the source and return electrode pads are applied to a patient a user may put the switch in a first position to create a first electrical path to communicate a first shock between the first and return electrodes via a first vector. If the first shock fails the user may move the switch to the second position to create a second electrical path to communicate a second shock between the second and return electrodes via a second vector. As a result, the system may help a layman easily flip a switch (or have an AED automatically flip the switch) to produce a first shock via a first vector and a second shock via a second vector—thereby increasing the chances of successfully treating a patient.

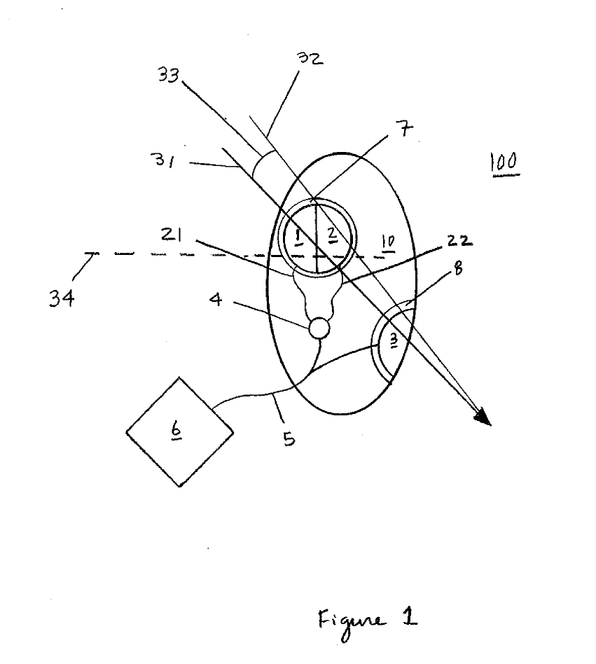

FIG. 1 includes cardiac resuscitation system 100 in an embodiment of the invention. Cardiac resuscitation system 100 may include external cardiac resuscitation device 6. External cardiac resuscitation device 6 may be any of various therapy devices including, without limitation, an AED, an external defibrillator, cardioverter, and an electrocardiogram (ECG) monitoring system (e.g., a device for monitoring ECG and/or pacing the heart), and the like. For purposes of explanation device 6 may be referred to herein as an AED but embodiments are not so limited.

System 100 may include first surface electrode 1 and second surface electrode 2. Electrodes 1, 2 may be included in a single contiguous source electrode pad 7. In an embodiment, electrodes 1, 2 are permanently and fixedly located at least 2 inches from one another within source electrode pad 7. Thus, a layperson can quickly apply a single electrode pad (pad 7) to the patient's chest and in the process, actually apply two different electrodes (electrodes 1, 2) to the patient. As explained below, these different electrodes will be used to generate different shocks, via different vectors, to patient 10. A “single contiguous” pad as used herein means the pad, when applied to the patient and ready to apply therapy to the patient, still includes both electrodes 1, 2 within a single pad (electrodes 1 and 2 have not been separated from one another because they are “fixed” within the pad and not designed for separation from one another). System 100 may also include return electrode 3 included in return electrode pad 8.

Potential pad placement patterns are unlimited and include, for example, anterior-posterior placement (where pad 7 is placed on the front of patient 10 and pad 8 is placed on the back of patient 10), sternal-apical placement (where pad 7 is placed on the front of patient 10 and pad 8 is placed on the patient's side below the arm), and the like. While 2 inches of separation between electrodes 1, 2 is used for ease of explanation, other embodiments are not so limited and include separations of 0.5, 1.0, 1.5, 2.5, 3.0, 3.5, 4.0, 4.5, 5.0, 5.5 inches and the like. The distance may be taken between the nearest points between outer edges of the electrodes.

System 100 may include a single electronic coupler 5 to electrically couple electrodes 1, 2 to a shared single therapeutic energy output of AED 6. Thus, a single shock from AED 6 may be delivered from a single AED output to coupler 5 (e.g., a cable, junction node, junction box, and the like) and then to switch 4, which is also included in the system and is used to direct shocks to electrodes 1 and/or 2. In an embodiment, coupler 5 includes not only a wire or cable coupling AED 6 to switch 4 but may also include a wire or cable coupling electrode 3 to AED 6. This way a possibly frantic layperson can attach a single coupler (e.g., a cable) with a keyed end into a reciprocally keyed junction (and possibly color coded) on AED 6. In other embodiments, switch 4 and electrode 3 may couple to AED 6 via separate couplers. Thus, embodiments including only the pad 7 and/or 8 may be supplied separately from any AED. A user may purchase only pad 7 and/or 8 knowing it will cooperate with a previously purchased AED (e.g., Lifepack 200 AED).

Switch 4, which is coupled to electrodes 1 and 2, includes first and second positions, herein referred to at times as positions #1 and #2. When the source and return electrode pads 7, 8 are applied to patient 10 and coupler 5 is coupled to AED 6, the system is set for delivering therapeutic energy to patient 10. When switch 4 is in position #1 a first electrical path is operable (via cable 21) to communicate therapeutic energy (e.g., an AED shock) between electrodes 1 and 3, via first vector 31, without being communicated to electrode 2. When switch 4 is in position 2 a second electrical path is operable (via cable 22) to communicate therapeutic energy between electrodes 2 and 3, via second vector 32, without being communicated to electrode 1.

In an embodiment, vector 31 includes a first direction and a first magnitude and vector 32 includes a second direction and a second magnitude; and the first direction is unequal to the second direction. For example, the first direction is unequal to the second direction by angle 33. Angle 33 may be 1, 2, 3, 4, 5, 6, 7, 8 or more degrees. Thus, by changing switch 4 between positions #1 and #2 a layperson (or medic, emergency responder, technician, nurse, physician, and the like) can easily apply two energy applications (e.g., shocks, pacing, etc.) via two different vectors (e.g., 31, 32) that are separate by, for example, 3 degrees. The change in vector may allow a second shock to succeed where a first shock failed—all without the added cost or time of using extra pads to deliver the second shock or relying on a frantic layperson to have the composure to set up a system for a second shock via a second vector.

As for bearings in determining vector degree shift angle 33, in one embodiment electrodes 1, 2 are located on the patient's chest and included in single axial plane 34 and the return electrode is located on the patient's side. In an embodiment the axial plane intersects the base of the heart. Vector 31 may be determined by following an imaginary line between the center-most points of electrodes 1, 3 and vector 32 may be determined by following an imaginary line between the center-most points of electrodes 2, 3 (noting FIG. 1 is not to scale and embodiments are not limited to the exact layout shown in FIG. 1).

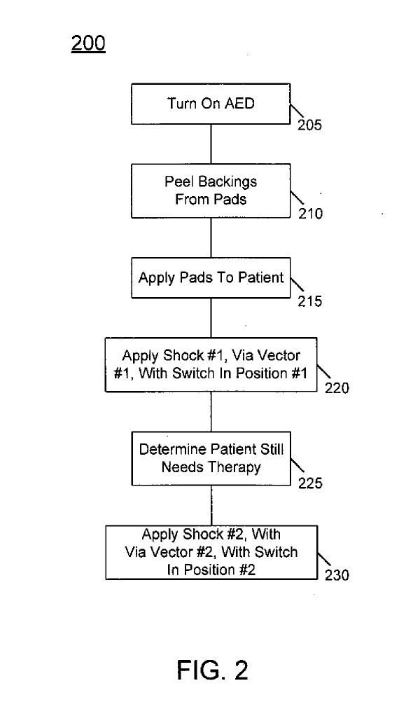

FIG. 2 includes method 200 for using a cardiac resuscitation system in an embodiment of the invention. In block 205 a user turns the AED device on. In blocks 210, 215 the user peels any backing from source and return pads and applies the pads to the patient. In block 220 the user may place switch 4 in position #1 and operate a “shock” button on the AED to apply a first shock (shock #1) to the patient via electrodes 1, 3 and vector 31 (vector #1). In block 225 the user may determine the patient still needs therapy (e.g., the patient is still experiencing an arrhythmia). In block 230 the user may place switch 4 in position #2 and operate a “shock” button on the AED to apply a second shock (shock #2) to the patient via electrodes 2, 3 and vector 32 (vector #2).

In various embodiments there may be various levels of automation. For example, AED 6 may include a processor and at least one non-transitory machine readable medium (e.g., flash memory) comprising instructions (e.g., code) that when executed on the processor included in AED 6, cause AED 6 to perform a method such as for various portions of method 200. For example, AED 6 may deliver first therapeutic energy to the patient via electrodes 1, 3. AED 6 may do this automatically once it monitors the patient, determines the patient has an arrhythmia, and gives warning to the user to step away from the patient in anticipation of a shock. AED 6 may further determine the patient still has an abnormal heart rhythm after delivering the first therapeutic energy to the patient. Then, based on determining the patient has an abnormal heart rhythm after delivering the first therapeutic energy to the patient, AED 6 may automatically switch switch 4 from position #1 to position #2 and then deliver second therapeutic energy to the patient via electrodes 2, 3. In some embodiments, the state of switch 4 may be remotely controlled based on, for example, the patient's heart rhythm (e.g., for one type of AED diagnosed arrhythmia choose switch position #1 and for another type of AED diagnosed arrhythmia choose switch position #1), the history of shocks previously administered (e.g., if the AED determines two shocks have already been applied via position #1 then switch to position #2 but if only one shock has been administered via position #1 then the next shock should again be applied via position #1), the size of shocks (e.g., if the previous shock at position #1 was less than 200 J then supply the next shock at position #1 at a level above 200 J), the impedance of each electrode (e.g., determine whether electrode 1 or 2 has the lower impedance and deliver a shock via the electrode that has the lower impedance), and other similar factors.

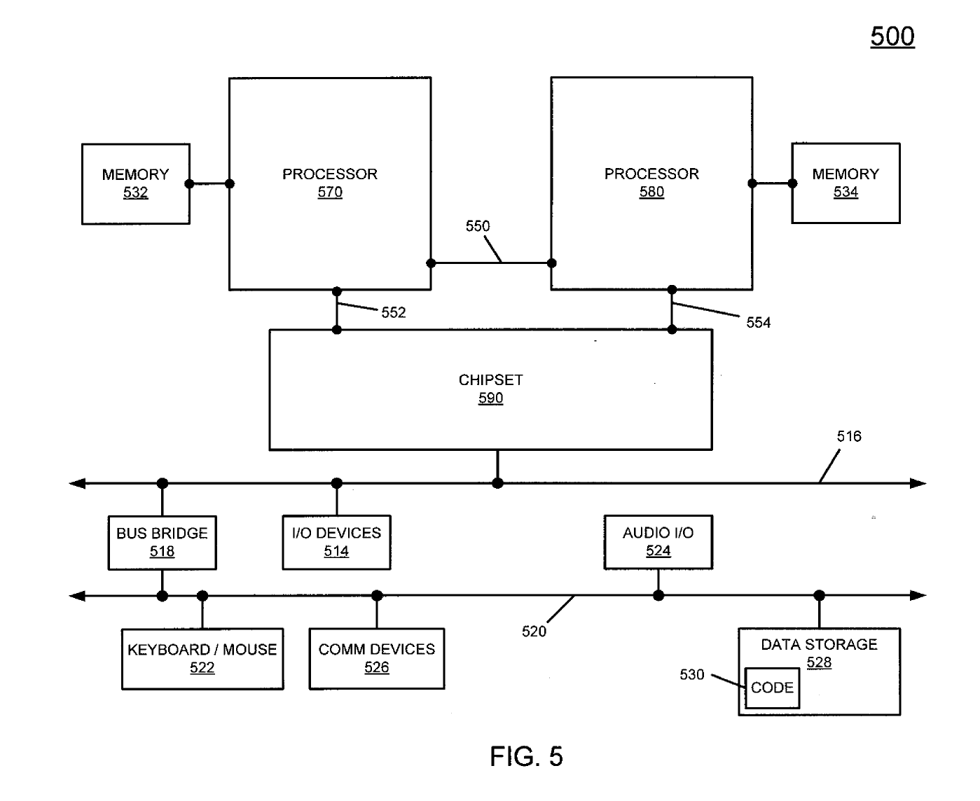

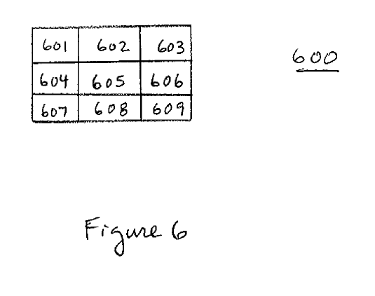

AED 6 may be implemented via various computer architectures and software packaging such as, for example, FIG. 5 (discussed further below). Of course, in some embodiments switch 4 is manually switchable, by a user, between positions #1 and #2.

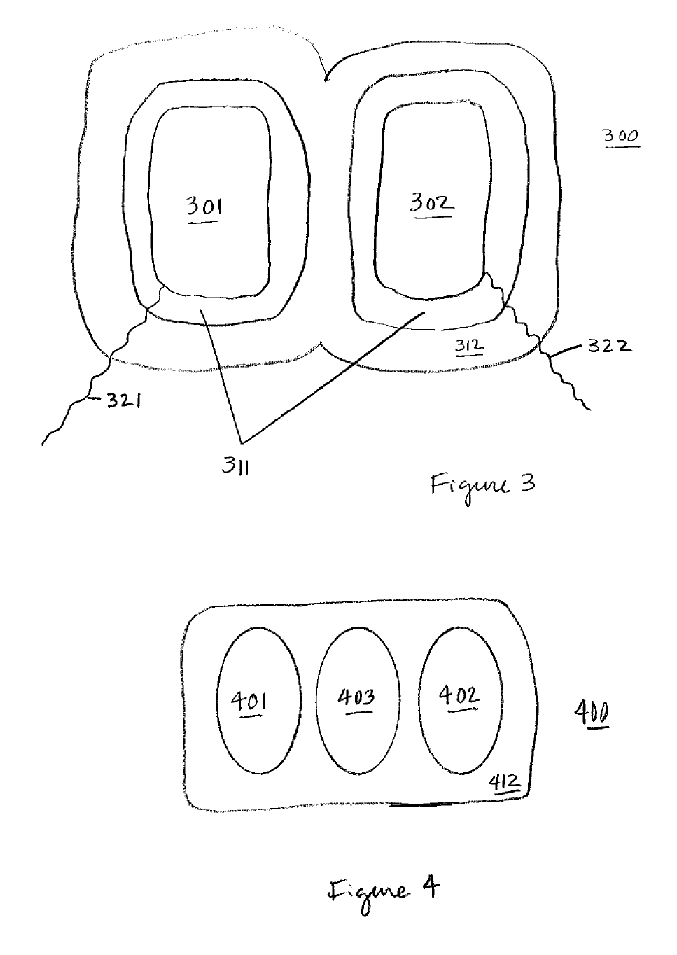

FIG. 3 includes an embodiment of a source electrode pad that could be used with system 100 of FIG. 1 or with other systems. Source pad 300 includes electrodes 301, 302 which can be used to respectively route energy via cables 321, 322 to a return pad (not shown) via different vectors. Pad 300 includes electrical insulator 311 electrically insulating (wholly or partially) electrodes 301 from electrode 302. Pad 300 further includes adhesive area 312 for adhering pad 300 to a patient. In various embodiments, pad juncture (between patient and pad) may be elastic, taped, or free floating.

In an embodiment, switch 4 may include a third position (position #3). In such an embodiment, when the switch is in position #3 first and second electrical paths are simultaneously operable to communicate therapeutic energy between electrodes 1, 3 via vector 31 and between electrodes 2, 3 via vector 32. Such an embodiment may therefore include operations modes that work in any or all of switch position #1 (current supplied to electrode 1 but not electrode 2), switch position #2 (current supplied to electrode 2 but not electrode 1), and position #3 (current supplied to electrodes 1 and 2).

FIG. 4 includes an electrode pad in an embodiment of the invention. Source electrode pad 400 includes electrodes 401, 402, 403. When switch 4 is in position #1 therapeutic energy is to be simultaneously communicated (a) between electrode 401 and a return electrode (not shown) via a first vector (extending from electrode 401 to the return electrode), (b) between electrode 403 and the return electrode via a third vector (extending from electrode 403 to the return electrode), and (c) without being supplied to electrode 402. When switch 4 is in position #2 therapeutic energy is to be simultaneously communicated (a) between electrode 402 and the return electrode via a second vector (extending from electrode 402 to the return electrode), (b) between electrode 403 and the return electrode via the third vector, and (iii) without being supplied to electrode 401.

In an embodiment, electrodes included in a source pad may be unevenly sized. For example, in FIG. 4 electrodes 401 and 402 may be evenly sized and larger than electrode 403. In such an embodiment current may be supplied in direct proportion to electrode size. For example, larger current may be supplied to electrodes 401, 402 than electrode 403. In an embodiment, low level resistors with high power ratings may be used to split current proportionally to the size of the pads. The short discharge time of the AED (e.g., 10 ms) may allow the use of common resistors that will not be damaged by the discharge. In FIG. 3, electrodes 301 and 032 may be unevenly sized. For example, electrode 301 may be larger than electrode 302 and receive more current than electrode 302. Of course, the same total amount of current may be supplied to evenly sized electrodes but with more concentrated current delivered via the smaller electrode.

Pads described herein may be used for monitoring, pacing, defibrillation, and the like. In an embodiment, a pad (e.g., pad 7 or 8) covers a surface area of 60, 70, 80, 90, 100, 110, 120, 130, 140, 150, 160, 170, 180 square cm and may include a diameter of, for example, 6, 7, 8, 9, 10, 11, 12, 13 cm diameters. In embodiment, each separate and distinct conductive electrode section (e.g., electrodes 1 and 2) each has an area of less than 150 square cm. The electrode shape may be rectangular, circular, and the like. The pad and/or electrode may have curved edges to avoid charge concentrations at the pad and/or electrode corners. Embodiments of the pad system (e.g., including pad 7 and/or pad 8) are compatible with existing AED units (via coupler 5) and meet Class III FDA standards for life-saving medical devices. Two electrode pads (e.g., FIGS. 1 and 3) and three electrode pads (e.g., FIG. 4) may each have a total area of 140 square cm. With FIG. 4, the effective area used for each shock would be the summed areas of the middle electrode and either side electrode.

In various of the above embodiments current has been described as originating in a single upper chest electrode (e.g., electrode 1 or 2) and returning to AED 6 via a second electrode (e.g., electrode 3), placed on the patient's side. However, the direction of current may be reversed and sent between any two electrodes or even generally sent from a single electrode with no designated return electrode.

In the embodiment of FIG. 6, more than two electrodes are supplied in the source electrode. Pad 600 includes electrodes 601, 602, 603, 604, 605, 606, 607, 608, 609. A switch may be used to direct current (manually or automatically) to any one of these electrodes. Also, current may be supplied simultaneously to any two or more of these electrodes, any three or more of these electrodes, and the like.

Also, source electrodes need not be located in a single pad. A single pad with multiple source electrodes is just one embodiment. Other embodiments may provide, for example, electrodes 1 and 2 in separate pads. An embodiment includes tear-away pads that could separate electrodes if necessary. For example, pad 300 of FIG. 3 could include a vertical perforation (not shown) between electrodes 301, 302 so the electrodes could be separated from each other as circumstances dictate (e.g., for an extremely large patient). Also, pads (e.g., pads 7 and 8) may be included in a vest worn by at an at-risk patient. The vest may tightly couple the pads to patient to ensure the patient can be quickly shocked and/or monitored as medical conditions dictate.

Various embodiments provide benefits. Minimal time is added to the therapy process while providing the advantage of a second vector. For example, with a manually operated switch a user may alternate vectors in seconds. Further, despite the technical concepts of using various vectors to treat or monitor the patient, the operation of the embodiments is easily understood and quickly accomplished by a layperson. Also, pads (e.g., pads 7 and 8) described above are cost effective. Embodiments are cost effective as many are compatible with present AED devices. Also, the ability to supply shocks via different shock vectors is less expensive than the cost of two conventional pads required for a reapplication of pads. Also, since the metal components of the pads wear over time (resulting in pad expiration after about two years), this upgrade in AED function could be done seamlessly during pad replacement, maximizing cost-effectiveness and convenience for the implementation of various embodiments to already deployed AED systems.

Embodiments provide an alternate vector for shock application following a failed initial attempt. By supplying this alternate vector, AED users without formal training will be able to continue treatment and provide more opportunities to defibrillate/cardiovert/treat the patient even after multiple failed shock attempts. Conventional AED systems do not offer the use of a second shock along a different vector, leading to a sub-optimal survival rate.

As mentioned above, misalignment of pads can result in a series of failed shocks. However, embodiments facilitate a quick change of the cardiac vector following failure of the initial shock application without a time consuming pad readjustment. For example, in an embodiment two electrodes are affixed onto one large pad. Having one large pad rather than multiple separate pads reduces complexity of application and increases the chances that at least one of the electrodes is in the proper position to produce an effective shock via a properly aligned vector. Additionally, conventional AEDs may inflict skin burns when the shock is applied along the same vector multiple times in succession, a problem that may be minimized by the application of an alternate vector. Since the energy of the shock conventionally increases with each successive application through the mechanism of the AED, the final applications often inflict the most severe burns. The secondary vector may be successful without requiring high intensity shocks and may help circumvent this problem.

Embodiments may include the following characteristics:

TABLE 1

| Criterion | Metric |
|---|---|
| Operation Time | <10 second increase in time to first shock |
| | compared to standard pad control group |
| | <2 second increase in time between first |
| | vector shock and second vector shock |
| Placement | 86% place correctly |
| Accuracy | |
| Withstands High | Switch can withstand 200 J shock through 4 |
| Voltage | successive shocks 2 minutes apart without |
| | device failure or malfunction |
| Secondary Pad | Switch successfully changes vector circuit |
| Success | |
| Significant | 3-15 degree change in vector |
| Vector Change | |
| Reduced Burns | <1 degree Celsius increase in temperature |
| Cost Effective | <$130 |

Regarding hardware, switch 4 may be capable of handling the current applied by an AED. One embodiment includes a switch rated to withstand over 2,000 V and 40 A, while other embodiments include switches rated at 250 V and 3 A, 125 V and 6 A, and capable of handling power up to 360 J over a 10 ms period of time. Embodiments may be suitable for patients of varying sizes (e.g., patients having surface areas of 1.6, 1.75, 1.9±0.15 square M). In an embodiment a switch is included directly on pad 7. Such a switch could slide between electrodes 1, 2. Embodiments include an automated electronic switch, a slider switch, a track switch, and plugging/unplugging system. The switches may create time delays of negligible length. In some embodiments the shock generated by an AED system is applied over 10 ms and consequently many low-cost switches are able to conduct the current over this short period of time without any damage to the switch. One embodiment includes a switching device comprising an IEPO toggle switch (Swi Togg) single pole double throw (SPDT) On-On heavy duty (20A) panel mount (66-1802) switch. In an embodiment the pads themselves have instructions printed upon them and display a diagram showing proper pad placement. The switching mechanism may be aligned with the corresponding pad sections providing intuitive operation (e.g., mount the switch along the vertical midline of pad 300 and toggle the switch to the left to send current via electrode 301 and toggle the switch to the right to send current via electrode 302). Wiring (e.g., cables 21, 22, 5) may be similar to cabling used in conventional AED devices.

In an embodiment a source electrode pad contains color coded A and B regions, with the first shock region as the blue A region and the second shock region as the green B region. The letters not only serve to signify which region is actively selected and in use, but also help to orient the pad on the patient correctly. There is also an image of the pads and how they should be placed correctly on a patient, using the pectoralis major muscle, nipples, clavicle, and sternum as major reference points for the AED operator. This image is meant to help the user identify landmarks on the body and align the pads to these landmarks. This may help align the electrodes along, above, or below axis 34. The instructions (e.g., written on the source pad) read “Flip switch from A to B after 2 unsuccessful shocks” to instruct the operator to change the switch at the appropriate time. Additional instructions (e.g., laminated instructional insert) may be included with the AED.

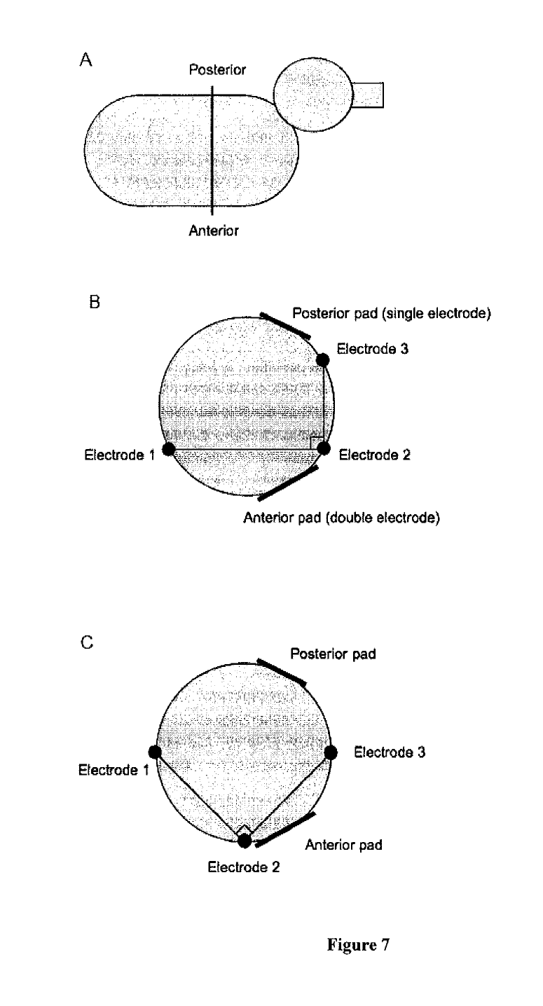

Table 2 below includes test data involving a three electrode system. A living pig body is used to model a human torso providing a sample at body temperature. Pads are placed on the porcine thoracic cavity, with electrode leads oriented in a triangle around the electrodes to create a voltage network similar to Einthoven's Triangle (see FIG. 7). The voltages measured at the electrodes are used to determine the direction of the current. By measuring the magnitude of the voltage along the two vectors created by the electrode system, the angle of applied current is calculated. Furthermore, a digital thermometer was used to measure the temperature of the pad areas immediately following 2 shocks at 30 second intervals at 200 J for each pad to assess potential burns that would occur on a patient. This temperature is compared to 4 shocks through a standard pad.

Mathematical modeling is used to quantify and optimize the change in angle. If the porcine cavity is brought to approximately the dimensions of a human thoracic cavity then computational modeling can be used to determine the angle change between the two vectors. The modeling program (Matlab™) accepts inputs of torso width and depth, distance from sternum for the two sternal electrodes, distance from the middle of the back for the apical electrode, and vertical distance between front and back electrodes. It then outputs the 3-dimensional angle change between the two vectors. This program models the torso as an elliptical prism, the electrodes as point sources of current, and current as existing linearly between these points. The qualitative test utilizing the electrode system, combined with the quantitative model, provides a value for the vector angle change provided by the device.

TABLE 8

| | Switch Left, | Switch Left, | Switch Right, | Switch Right, |
|---|---|---|---|---|
| | Electrodes | Electrodes | Electrodes | Electrodes |
| | 1-2 | 2-3 | 1-2 | 2-3 |
| Test 1 | 36.8 ± 0.55 | 30.6 ± 1.19 | 37.1 ± 1.15 | 26.9 ± 0.34 |
| Test 2 | 34.2 ± 0.65 | 27.5 ± 0.61 | 35.8 ± 0.96 | 25.6 ± 1.13 |

| | Angle, Switch Left | Angle, Switch Right | Angle Difference |
|---|---|---|---|
| Test 1 | 39.8° | 35.9° | 3.88° |
| Test 2 | 38.8° | 35.6° | 3.27° |

Referring now to FIG. 5, shown is a block diagram of a system in accordance with an embodiment of the present invention. Multiprocessor system 500 is a point-to-point interconnect system, and includes a first processor 570 and a second processor 580 coupled via a point-to-point interconnect 550. Each of processors 570 and 580 may be multicore processors. The term “processor” may refer to any device or portion of a device that processes electronic data from registers and/or memory to transform that electronic data into other electronic data that may be stored in registers and/or memory.

First processor 570 may include a memory controller hub (MCH) and point-to-point (P-P) interfaces. Similarly, second processor 580 may include a MCH and P-P interfaces. The MCHs may couple the processors to respective memories, namely memory 532 and memory 534, which may be portions of main memory (e.g., a dynamic random access memory (DRAM)) locally attached to the respective processors. First processor 570 and second processor 580 may be coupled to a chipset 590 via P-P interconnects, respectively. Chipset 590 may include P-P interfaces.

Furthermore, chipset 590 may be coupled to a first bus 516 via an interface. Various input/output (I/O) devices 514 may be coupled to first bus 516, along with a bus bridge 518, which couples first bus 516 to a second bus 520. Various devices may be coupled to second bus 520 including, for example, a keyboard/mouse 522, communication devices 526, and data storage unit 528 such as a disk drive or other mass storage device, which may include code 530, in one embodiment. Further, an audio I/O 524 may be coupled to second bus 520.

Embodiments may be implemented in code and may be stored on a storage medium having stored thereon instructions which can be used to program a system to perform the instructions. The storage medium may include, but is not limited to, any type of disk including floppy disks, optical disks, optical disks, solid state drives (SSDs), compact disk read-only memories (CD-ROMs), compact disk rewritables (CD-RWs), and magneto-optical disks, semiconductor devices such as read-only memories (ROMs), random access memories (RAMs) such as dynamic random access memories (DRAMs), static random access memories (SRAMs), erasable programmable read-only memories (EPROMs), flash memories, electrically erasable programmable read-only memories (EEPROMs), magnetic or optical cards, or any other type of media suitable for storing electronic instructions.

Embodiments of the invention may be described herein with reference to data such as instructions, functions, procedures, data structures, application programs, configuration settings, code, and the like. When the data is accessed by a machine, the machine may respond by performing tasks, defining abstract data types, establishing low-level hardware contexts, and/or performing other operations, as described in greater detail herein. The data may be stored in volatile and/or non-volatile data storage. For purposes of this disclosure, the terms “code” or “program” cover a broad range of components and constructs, including applications, drivers, processes, routines, methods, modules, and subprograms. Thus, the terms “code” or “program” may be used to refer to any collection of instructions which, when executed by a processing system, performs a desired operation or operations. In addition, alternative embodiments may include processes that use fewer than all of the disclosed operations, processes that use additional operations, processes that use the same operations in a different sequence, and processes in which the individual operations disclosed herein are combined, subdivided, or otherwise altered.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. A cardiac resuscitation system comprising:
first and second surface electrodes included in a single contiguous source electrode pad and a return electrode included in a return electrode pad;
a single electronic coupler to electrically couple the first and second electrodes to a shared single therapeutic energy output of an external cardiac resuscitation device; and
a switch, coupled to the first and second electrodes, including first and second positions;
wherein when the source and return electrode pads are applied to a patient and the switch is in (a) the first position a first electrical path is operable to communicate therapeutic energy through and between the first and return electrodes, via a first vector, without being communicated through the second electrode, and (b) the second position a second electrical path is operable to communicate therapeutic energy through and between the second and return electrodes, via a second vector, without being communicated through the first electrode.

2. The system of claim 1, wherein the first and second electrodes are permanently and fixedly located at least 2 inches from one another within the source electrode pad.

3. The system of claim 2, wherein:
the first vector includes a first direction and a first magnitude;
the second vector includes a second direction and a second magnitude; and
the first direction is unequal to the second direction.

4. The system of claim 3, wherein the first direction is unequal to the second direction by at least 3 degrees.

5. The system of claim 3, wherein the first direction is unequal to the second direction by at least 3 degrees when the first and second electrodes are located on the patient's chest and in a single axial plane and the return electrode is located on the patient's side.

6. The system of claim 3, wherein the source electrode pad includes an electrical insulator electrically insulating the first electrode from the second electrode.

7. The system of claim 1, wherein:
the switch includes a third position; and
when the switch is in the third position the first and second electrical paths are simultaneously operable to communicate therapeutic energy through and between the first and return electrodes via the first vector and through and between the second and return electrodes via the second vector.

8. The system of claim 1 comprising the external cardiac resuscitation device, wherein the external cardiac resuscitation device includes one of an automated external defibrillator (AED), an external defibrillator, and an electrocardiogram monitoring system.

9. The system of claim 8 comprising at least one non-transitory machine readable medium comprising instructions that when executed on a processor, which is included in the external cardiac resuscitation device, cause the external cardiac resuscitation device to perform a method comprising:
delivering first therapeutic energy to the patient through and between the first and return electrodes;
determining the patient has an abnormal heart rhythm after delivering the first therapeutic energy to the patient;
based on determining the patient has an abnormal heart rhythm after delivering the first therapeutic energy to the patient, (i) automatically switching the switch from the first position to the second position, and then (ii) delivering second therapeutic energy to the patient through and between the second and return electrodes.

10. The system of claim 1, wherein:
the source electrode pad includes a third electrode;
when the switch is in the first position therapeutic energy is to be simultaneously communicated (c)(i) through and between the first and return electrodes via the first vector, (ii) through and between the third and return electrodes via a third vector, and (iii) without being supplied through the second electrode; and
when the switch is in the second position therapeutic energy is to be simultaneously communicated (d)(i) through and between the second and return electrodes via the second vector, (ii) through and between the third and return electrodes via the third vector, and (iii) without being supplied through the first electrode.

11. The system of claim 10, wherein when the switch is in the first position therapeutic energy is to be unevenly communicated through and between the first and return electrodes and the third and return electrodes.

12. The system of claim 1, wherein the switch is manually switchable, by a user, between the first and second positions.

13. A cardiac resuscitation system comprising:
first and second surface electrodes;
a single electronic coupler to electrically couple the first and second electrodes to a shared single therapeutic energy output of an external cardiac resuscitation device; and
a switch, coupled to the first and second electrodes, including first and second positions;
wherein when the first and second surface electrodes are applied to a patient and the switch is in (a) the first position a first electrical path is operable to communicate therapeutic energy through and between the first electrode and a return electrode, via a first vector, without being communicated through the second electrode, and (b) the second position a second electrical path is operable to communicate therapeutic energy through and between the second and return electrodes, via a second vector, without being communicated through the first electrode.

14. The system of claim 13, wherein:
the switch includes a third position; and
when the switch is in the third position the first and second electrical paths are simultaneously operable to communicate therapeutic energy through and between the first and return electrodes via the first vector and through and between the second and return electrodes via the second vector.

15. The system of claim 13, comprising the external cardiac resuscitation device and at least one non-transitory machine readable medium comprising instructions that when executed on a processor, which is included in the external cardiac resuscitation device, cause the external cardiac resuscitation device to perform a method comprising:
delivering first therapeutic energy to the patient through and between the first and return electrodes;
determining the patient has an abnormal heart rhythm after delivering the first therapeutic energy to the patient;
based on determining the patient has an abnormal heart rhythm after delivering the first therapeutic energy to the patient, (i) automatically switching the switch from the first position to the second position, and then (ii) delivering second therapeutic energy to the patient through and between the second and return electrodes.

16. The system of claim 13, comprising a source electrode pad that includes a third electrode and the first and second electrodes; wherein:
when the switch is in the first position therapeutic energy is to be simultaneously communicated (c)(i) through and between the first and return electrodes via the first vector, (ii) through and between the third and return electrodes via a third vector, and (iii) without being supplied through the second electrode; and
when the switch is in the second position therapeutic energy is to be simultaneously communicated (d)(i) through and between the second and return electrodes via the second vector, (ii) through and between the third and return electrodes via the third vector, and (iii) without being supplied through the first electrode.

17. At least one machine readable medium comprising instructions that when executed on a computing device cause the computing device to perform a method comprising:
delivering first therapeutic energy to a patient through and between a first surface electrode and a return electrode along a first vector;
determining the patient has an abnormal heart rhythm after delivering the first therapeutic energy to the patient;
based on determining the patient has an abnormal heart rhythm after delivering the first therapeutic energy to the patient, (i) automatically switching a switch from a first position to a second position, and then (ii) delivering second therapeutic energy to the patient through and between a second surface electrode and the return electrode along a second vector:
wherein the first and second surface electrodes are included in a single contiguous source electrode pad and the return electrode is included in a return electrode pad.

18. The at least one medium of claim 17, wherein the first and second electrodes are permanently and fixedly located at least 2 inches from one another within the source electrode pad.

19. The at least one medium of claim 17, wherein:
the switch includes a third position; and
the method comprises when the switch is in the third position simultaneously communicating therapeutic energy through and between the first and return electrodes via the first vector and through and between the second and return electrodes via the second vector.
